(19) United States
(12) Patent Application Publication
(10) Pub. No.: US 2024/0300874 A1
(43) Pub. Date: Sep. 12, 2024

(54) PROCESS FOR EXTRACTING LIGHT HYDROCARBONS FROM AGGREGATE MATERIAL, INCLUDING LRA AND OTHER AGGREGATE

(71) Applicant: Tyche Extraction Company, LLC, Lakeway, TX (US)

(72) Inventor: Billy Shane McDade, Lakeway, TX (US)

(21) Appl. No.: 18/600,136

(22) Filed: Mar. 8, 2024

Related U.S. Application Data
(60) Provisional application No. 63/489,031, filed on Mar. 8, 2023.

(51) Int. Cl. C07C 7/10; C07C 7/00; C07C 7/148

(52) U.S. Cl. CPC C07C 7/10; C07C 7/005; C07C 7/148

(57) ABSTRACT

Disclosed is method that involves subjecting a base material to an extraction process to extract hydrocarbon fractions having molecular weights within a desired range from the base material to generate a resultant extraction material comprising mostly if not entirely of hydrocarbon fractions having molecular weights within the desired range. In some embodiments, the extraction process can involve performing the extraction in iterations.

### CROSS-REFERENCE TO RELATED APPLICATIONS

This patent application is related to and claims the benefit of priority to U.S. provisional patent application No. 63/489,031, filed on Mar. 8, 2023, the entire contents of which is incorporated herein by reference.

### FIELD OF THE INVENTION

Embodiments can relate to processes for extracting hydrocarbons from material, and in particular processes for extracting specific ranges of hydrocarbon molecular weights from material.

### BACKGROUND OF THE INVENTION

Many naturally occurring material includes hydrocarbon compounds that can be extracted. This can be done to process or condition the material in a desired way and/or to use the extracted hydrocarbons for other processes. Known methods for extracting hydrocarbon compounds can be inefficient, ineffective, and not cost-effective, especially when it involves extracting a certain line of hydrocarbon fraction compounds (e.g., type, amount, range, and/or concentration of hydrocarbon molecular weights). Thus, conventional methods can be used to extract the hydrocarbons and other compounds and minerals, but these conventional methods are limited in that they cannot successfully extract a specific line of hydrocarbon fractions in a manner that is economically and commercially sustainable.

One of the ways to enhance extracting hydrocarbons from material is by freeing or loosening the hydrocarbon fractions within the matrix of the material. Known techniques for free or loosening hydrocarbon fractions can be appreciated from U.S. Pat. No. 10,961,462, the entire contents of which is incorporated herein by reference in its entirety. Embodiments of the inventive method disclosed herein improve upon techniques disclosed in U.S. Pat. No. 10,961,462.

### SUMMARY OF THE INVENTION

Embodiments of the inventive method can involve subjecting material to an extraction process to extract a certain line of hydrocarbon fraction compounds (e.g., type, amount, range, and/or concentration of hydrocarbon molecular weights) from the material to generate a resultant extracted material. It is contemplated for the material to include hydrocarbon fractions having molecular weights within the range from C1 to C60. Thus, the extraction process can be used to remove certain amounts of hydrocarbon fractions so that the resultant material is completely devoid of those hydrocarbon fractions, substantially devoid of those hydrocarbon fractions, or has a predetermined concentration of those hydrocarbon fractions remaining therein. Similarly, the extraction process can generate an extracted material containing the extracted hydrocarbons. This extracted material can consist of these hydrocarbon fractions, consist essentially of these hydrocarbon fractions, or have a predetermined concentration of these hydrocarbon fractions contained therein. As can be appreciated, the inventive method can be used to tailor which type of hydrocarbon fraction(s) (e.g., which molecular weight(s)) and/or the amount(s) to degree(s) which witch it/they are extracted.

The material from which extraction is performed can be material typically used as aggregate in a construction material process (e.g., aggregate for making asphalt concrete, aggregate for making Portland cement concrete, aggregate for making clinker, etc.), but it need not be. The material from which extraction is performed can be waste material or a by-product material, but it need not be. For instance, while the inventive method may be tailored to provide the most efficiencies and the most cost-effectiveness when the material is waste material or a by-product material of the production of construction aggregates, the inventive method is effective when used to process any type of hydrocarbon-containing material, regardless of whether it is waste material or a by-product material and regardless of whether it is intended to be used as a construction material aggregate.

It is contemplated for the material from which extraction is performed to be bituminous material (e.g., material containing bitumen), but it need not be. It is contemplated for the material to contain hydrocarbons with hydrocarbon fractions within a range from C1 to C60, but it understood that the material can contain any amount or range of hydrocarbon fractions. A preferred material can be Limestone Rock Aggregate (“LRA”). LRA is a naturally occurring limestone material that is formed when a limestone deposit is naturally impregnated with various bituminous components, including asphaltenes and lighter hydrocarbons. LRA has been mined for many years, and can be processed into products used for roadway construction and maintenance, for example. Generally, during the processing (typically crushing) of stone aggregate material, a by-product material is produced which is commonly referred to as crusher fines. When LRA is processed, the by-product material is commonly referred to as LRA crusher fines. Embodiments of the inventive method can be tailored to perform extraction on LRA and/or LRA crusher fines.

An exemplary embodiment can relate to a method for extracting hydrocarbon fractions from a material. The method can involve subjecting a base material comprising hydrocarbon fractions to an extraction process, the extraction process involving a treatment configured to free or loosen hydrocarbon factions from a matrix of the base material, the treatment generating volatiles comprising hydrocarbon fractions within a desired range of molecular weights. The method can involve allowing the volatiles to enter a separator for separating the hydrocarbon fractions having molecular weights with the desired range of molecular weights from other components of the volatiles to generate a resultant extracted material.

The treatment can involve a heat treatment, a mechanical treatment, and/or a chemical treatment to impart energy to the base material for freeing or loosening the hydrocarbon fractions.

In some embodiments, the base material can include Limestone Rock Aggregate (“LRA”). In some embodiments, the base material can consist essentially of LRA. In some embodiments, the base material can consist of LRA. In some embodiments, the base material can include LRA crusher fines. In some embodiments, the base material can consist essentially of LRA crusher fines. In some embodiments, the base material can consist of LRA crusher fines.

In some embodiments, the base material can comprise hydrocarbon fractions having molecular weights within a first range. The resultant extracted material can comprise hydrocarbon fractions having molecular weights within a second range. The first range can be greater than the second range. For instance, the first range can be from C1 to C60. The second range can be a sub-range of the first range—e.g., the sub-range can be C1 to C14 for example.

In some embodiments, the method can involve adjusting the treatment to modulate the desired range of molecular weights. For instance, the method can involve performing the extraction process in iterations to modulate the desired range of molecular weights. As an example, the extraction process can be implemented via a first iteration to free/loosen hydrocarbons fractions of a specific molecular weight or ranges of weights, and then implanted in a second iteration to free/loosen hydrocarbon fractions of a different specific molecular weight or ranges of weights, etc. As another example, the method can involve modulating the desired range of molecular weights by adjusting an amount and a duration of energy imparted to the base material—i.e., the amount and/or duration of energy imparted can modulate the ranges of molecular weights that are freed/loosened. For example, the amount and/or duration of energy imparted can facilitate freeing/loosening hydrocarbons fractions of C1, hydrocarbon fractions of C2, hydrocarbon fractions of C3, etc. or hydrocarbon fractions within a range of C1-C2, hydrocarbon fractions within a range of C1-C10, hydrocarbon fractions within a range of C13-C17, hydrocarbon fractions within a range of C40-C50, etc. It should be understood that the amount and/or duration of energy can be used to target a hydrocarbon fraction or range of hydrocarbon fractions, but the targeting it from light to heavy. For instance, the energy imparted would generally always free/loosen the lighter fractions and depending on the amount or duration will also target the heavier fractions. Thus, one can use a first amount/duration to target C1-C5, for example, and then a second amount/duration to target C6-C10. Generally, it would be more economical to just use the second amount/duration to target the C1-C10, as that would free/loosen C1-C10 but it is possible to use the first amount/duration to target the C1-C5 and then use a second amount/duration to target C6-C10. For the sake of clarity, one could not merely use the second amount/duration to only target C6-C10, as the second amount/duration would free/loosen C1-C10. As another example, the method can involve modulating the desired range of molecular weights by selecting two or more of the heat treatment, the mechanical treatment, and the chemical treatment to be part of the treatment. For instance, a chemical treatment for a particular application might be better suited (e.g., more effective, more efficient, etc.) at freeing/loosening hydrocarbon fractions within a range from C1-7, whereas a mechanical treatment for the particular application might be better suited for freeing/loosening hydrocarbons within a range of C7-C15. Should it be desired to have a conditioned based material with only C1-C15 hydrocarbon fractions extracted therefrom, then one could modulate the ranges to achieve the same. As another example, the method can involve modulating the desired range of molecular weights by using one form of treatment before the other form of treatment. For instance, a heat treatment might be work for freeing/loosening hydrocarbons within a range of C1-C40, whereas a mechanical treatment is well suited for freeing/loosening hydrocarbons within a range of C1-C14 but it would be more energy efficient if the heat treatment were to be applied first and then the mechanical treatment to generate a conditioned based material with C1-C14 removed therefrom.

It is understood that the method can involve modulating the desired range of molecular weights by: 1) selecting two or more of the heat treatment, the mechanical treatment, and the chemical treatment to be part of the treatment; 2) using one form of treatment before the other form of treatment; and/or 3) adjusting an amount and a duration of energy imparted to the base material. There can be plural modulation techniques for one bac material. For instance, a first modulation technique can be used to target a first molecular weight or a first range of molecular weights. A second modulation technique can target a second molecular weight or a second range of molecular weights. The first modulation technique can free/loosen a first amount of hydrocarbon fractions. The second modulation technique can free/loosen a second amount of hydrocarbon fractions. The first amount of hydrocarbon fractions can be different from the second amount of hydrocarbon fractions.

Further features, aspects, objects, advantages, and possible applications of the present invention will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present innovation will be more apparent from the following more particular description thereof, presented in conjunction with the following drawings. Like reference numbers used in the drawings may identify like components.

FIG. 1 is an exemplary illustration of an embodiment of the extraction process (using heat, chemical, and/or mechanical energy) that can be used to extract light hydrocarbons from material.

FIG. 2 is an exemplary system configuration that can be used to carry out an embodiment of the extraction process.

FIGS. 3-4 are images of resultant extracted material being extracted by an embodiment of the extraction process.

FIG. 5 is an image of LRA crusher fines that have had the light hydrocarbon fractions extracted. The lighter colored material is the LRA crusher fines after the light hydrocarbon fractions had been removed by an embodiment of the extraction process. The darker colored material is unprocessed LRA crusher fines.

FIGS. 6-14 are laboratory test results of resultant extracted material that has been generated using an embodiment of the extraction process.

FIGS. 15-17 are laboratory test results of resultant extracted material, showing chromatographic analyses, crude oil fingerprint, and bio-marker summary information for drill cuttings obtained from closed loop extraction drilling methods (closed loop extraction generates dryer material).

FIGS. 18-20 are laboratory test results of resultant extracted material, showing chromatographic analyses, crude oil fingerprint, and bio-marker summary information for drill cuttings obtained from wet cutting extraction drilling methods (wet cutting extraction generates wetter material).

FIGS. 21-23 are laboratory test results of resultant extracted material, showing chromatographic analyses, crude oil fingerprint, and bio-marker summary information for left still material.

### DETAILED DESCRIPTION OF THE INVENTION

The following description is of exemplary embodiments that are presently contemplated for carrying out the present invention. This description is not to be taken in a limiting sense, but is made merely for the purpose of describing the general principles and features of the present invention. The scope of the present invention is not limited by this description.

Conventional methods for extracting hydrocarbons from material tend to focus on extracting all of the hydrocarbon or bituminous elements that may be present during the extraction process. Embodiments of the inventive process, however, can involve extraction of hydrocarbons from base material so that the only hydrocarbons extracted pertain to a specific molecular weight or a line of molecular weights, or that a majority of the hydrocarbons extracted pertains to a specific molecular weight or a line of molecular weights, or that a certain amount of one molecular weight or line of molecular weights is extracted while different amount of another molecular weight or line of molecular weights is extracted, etc.

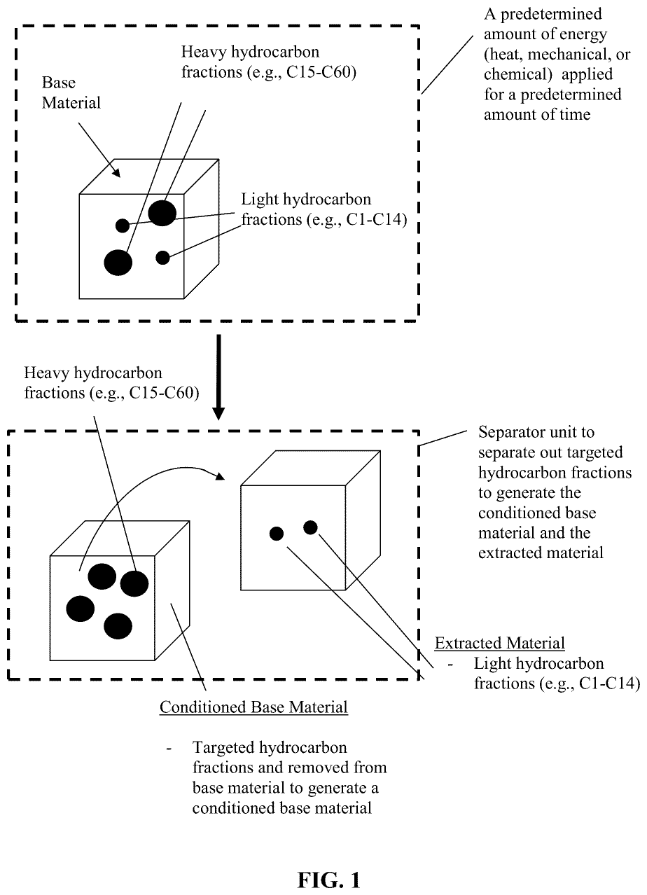

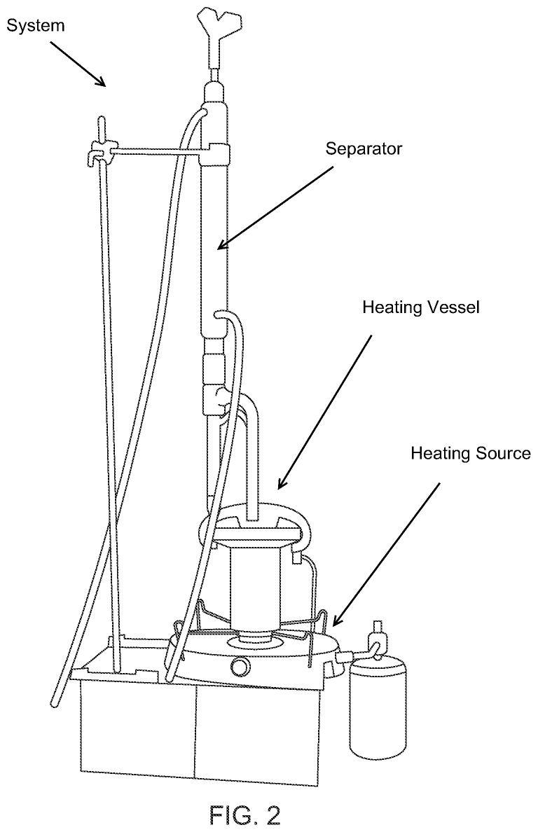

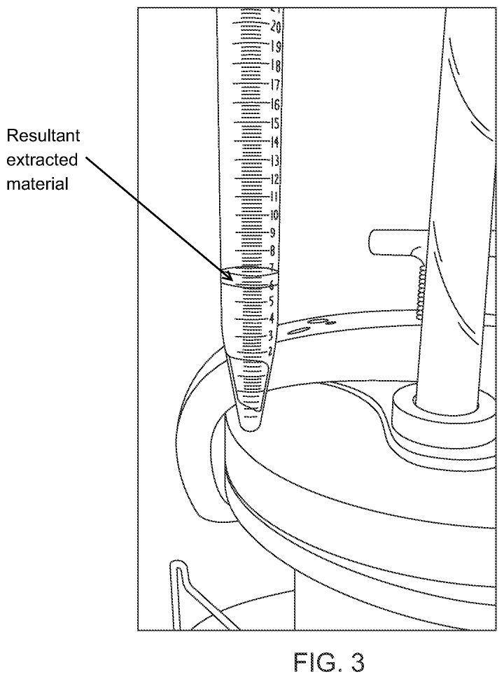

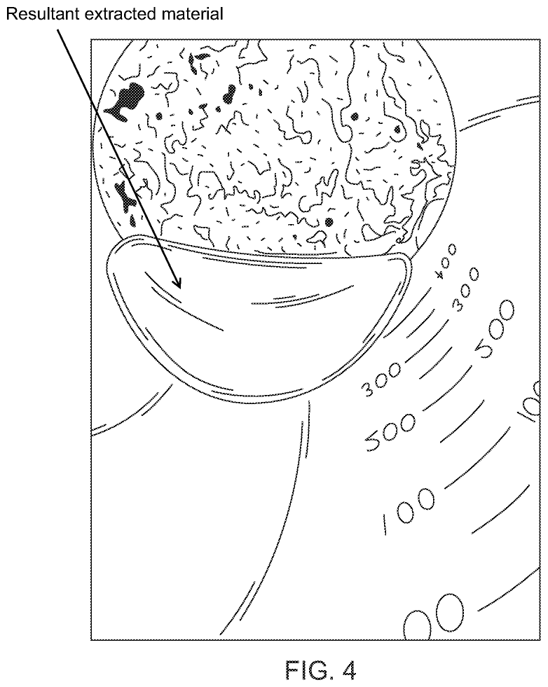

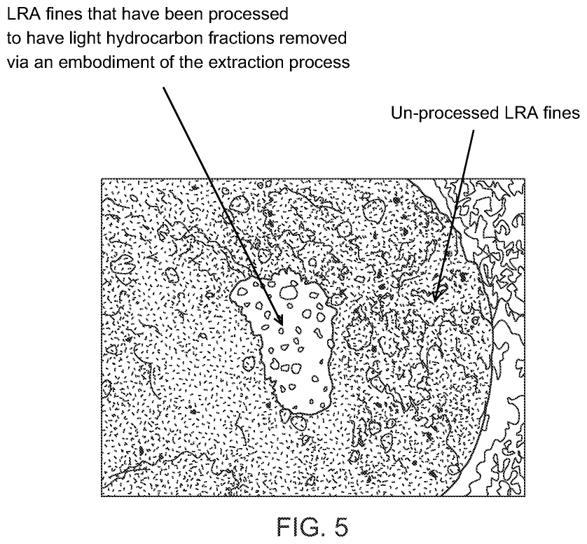

Referring to FIGS. 1-5, embodiments of the inventive method can involve subjecting base material to an extraction process. The base material can be naturally occurring material or non-naturally occurring material containing hydrocarbons, naturally occurring material that has hydrocarbons impregnated within it via natural processes, etc. The base material can include hydrocarbons having hydrocarbon fractions within the range from C1 to C60, for example. Hydrocarbon fractions having molecular weights from C1 to C14 can be referred to as light hydrocarbon fractions. Hydrocarbon fractions having molecular weights from C15 to C60 can be referred to as heavy hydrocarbon fractions. The base material can be bituminous material. The base material can be Limestone Rock Aggregate (“LRA”), LRA crusher fines, reclaimed or recycled asphalt (e.g., recycled asphalt pavement (“RAP”), etc. Some base materials can include a combination of these or other hydrocarbon containing materials.

The base material can be treated using the inventive method. The treatment of the base material can involve removal, or extraction, of certain bituminous material (e.g., light hydrocarbons, heavy hydrocarbons, etc.) from the base material). The treated base material and/or the extracted bituminous material can be used in other material processes. The treatment process can be designed to specifically extract hydrocarbon fractions from the base material in a desired range beginning with C1 and ending at the most advantageous hydrocarbon fraction (C2, C3, etc). This is accomplished through the modulation of the energy types (e.g., heat, mechanical, chemical), and their duration, described herein. “Advantageous”, as it is used here, can have several meanings, including but not limited to the range of extracted hydrocarbons that are most valuable to a hydrocarbon refinery, the range of hydrocarbons that would have value to a refinery but are more valuable left in the conditioned base material for some future use, or a combination of both (specifically viewed through a context of current market conditions). The treatment process can generate a conditioned base material (e.g., material that has had desired hydrocarbons extracted therefrom) and generate an extraction material (e.g., the extracted hydrocarbons). The conditioned base material can be optimized (e.g., the desired type and amount of hydrocarbon fractions removed therefrom) for use as an aggregate or use as a component in a subsequent material process, such as the manufacture of Portland cement, or to be further processed for the removal of additional valuable minerals or elements. The extracted material can be optimized only for its value to a hydrocarbon refinery, or may be adjusted such that it leaves the conditioned material with certain qualities such as those described above, even if it reduces the value of the hydrocarbon extracts to the refinery.

The extraction process can involve freeing or loosening hydrocarbon fractions from the matrix of the base material. A technique for freeing or loosening the hydrocarbon fractions from the matrix of the base material is briefly summarized by adding energy to small base material particles, which frees or loosens the hydrocarbon fractions and allows for their removal. Generally, the more energy that is applied, the more and/or heavier (e.g., heavier in molecular weight) the freed/loosened hydrocarbons become. The energy applied to the base material for hydrocarbon extraction can be heat energy, mechanical energy, chemical energy (e.g., use of a hydrocarbon rich solvent or a surfactant), or some combination of all three. These forms of applied energy can be referred to as treatments to the base material—i.e., the base material is treated by having energy applied to it to free/loosen a targeted hydrocarbon fraction(s). Known techniques for freeing/loosening hydrocarbon fractions can be appreciated from U.S. Pat. No. 10,961,462. Embodiments of the inventive method disclosed herein improve upon techniques disclosed in U.S. Pat. No. 10,961,462.

The energy imparted to the base material frees/loosens the hydrocarbon fractions from the matrix of the base material. Selection of which treatment and adjustment(s) to the energy applied can be done to control which hydrocarbon fraction(s) is/are freed/loosened from the base material, how much of or to what degree the hydrocarbon fraction(s) is/are freed/loosened from the base material matrix, etc. Some treatments might damage hydrocarbon fractions, some treatments are most cost effective, some treatments are more energy intensive, some treatments are more precise, etc. so the selection and adjustment may be done to implement tradeoffs between efficiency, effectiveness, cost, etc. Hydrocarbon fractions having molecular weights from C1 to C60 (or any range there-between) can be freed/loosened. Energy can be imparted to free/loosen the desired range (up to the desired molecular weight) and amount of hydrocarbon fraction(s) from the base material.

The conditioned based material and/or the extracted material may include other compounds (e.g., sulfur compounds, minerals, elements, etc.). While the inventive method may not be used to remove these other compounds (except in the sense that it is separating them at a desired rate from each other), the inventive method can be used to generate a conditioned base material and/or an extracted material that is better suited for a process that is designed to remove these compounds. In other words, operational and functional aspects of the inventive method can not only generate an extracted material with desired hydrocarbon fractions but it can also optimize that material for further processing (typically at a crude oil refining facility). Similarly, the process can be optimized such that the conditioned base material can most effectively be used for further processing, such as for a Portland cement feedstock, or the further removal of valuable elements or compounds. It is envisioned that optimization for the hydrocarbon extract and optimization for the conditioned base material might sometimes be mutually exclusive, and the operator must decide which optimized product is most valuable.

As will be demonstrated herein, the inventive method can be used to specifically tailor the removal of one or more specific hydrocarbon molecular weights from the base material in a very accurate, precise, efficient, and/or cost-effective manner. This can be done to generate a conditioned base material and/or an extracted material with a specific line of hydrocarbon(s) (e.g., type, amount, and/or concentration of hydrocarbon(s) up to a desired molecular weight). The inventive method uses mechanical, heat, and/or chemical processes (or variations thereof) to generate an optimal conditioned based material and/or an optimal extracted material. Optimal can mean optimal material properties for use as an aggregate, optimal cost-effectiveness for use as an aggregate or a material in a material process, optimal material properties for a waste product or a by-product, optimal cost-effectiveness for use as a material in a material processes, optimal material properties for extracting other compounds, minerals, or elements from the extracted material, optimal environmental impact, etc.

One of the ways to impart energy on the base material is to apply heat. Another way is via a chemical process. Another way is via mechanical processes (e.g., particle collision, application of pressure, high-sheer agitation, etc.). Embodiments can involve the use of any one or combination of heat, mechanical, or chemical means to free/loosen the hydrocarbon fractions from the matrix. The selection of which one or combination of energy-imparting method to use can depend on design criteria, trade-offs, cost-benefit analyses, such as cost, efficiency, type of material used, desired material properties of the resultant (hydrocarbon or conditioned base) material, etc. When used in combination, the chemical/heating/mechanical treatment(s) can be used before, during, and/or after the other form of treatment—i.e., the chemical treatment can be performed before, during, and/or after the mechanical treatment, etc. Which treatment to use, when to use it, the duration of use, etc. can be determined based on its effectiveness, efficiency, and accuracy in targeting a specific hydrocarbon fraction or a line of hydrocarbon fractions. This can also depend on the type of base material used. Some forms of treatment are more cost effective but are not as precise, some are effective for freeing/loosening a certain line of hydrocarbons but also damage another line of hydrocarbons, etc. Thus, the interplay of each of these three treatment processes is that use of them can be used as levers to control the overall process. As a non-limiting example, heat treatment can be configured to operate in a gross-like manner but can be efficient to target large ranges of hydrocarbon fractions. Mechanical treatment can be configured to operate in a more precise manner to target a specific hydrocarbon fraction but can be more expensive or energy intensive.

For the application of heat, the heat can be applied directly to the base material, the heat can be applied to a heat medium (e.g., a fluid, a gas, etc.) wherein the medium is then applied to the base material, or a combination of both. An exemplary heat medium can be water. In some embodiments, the heat required to free/loosening the hydrocarbon fractions from the matrix can be reduced if the heat medium is used.

An exemplary means to impart energy to free/loosening the hydrocarbon fractions from the matrix via chemical processes is through the use of a solvent, wherein the solvent, which when applied, can form a hydrocarbon rich solvent solution that is free from the matrix of the base material. In addition, or in the alternative, the base material and/or the hydrocarbon rich solvent solution can be subjected to a heating treatment to free or loosen hydrocarbon fractions from the matrix of the base material. In addition, or in the alternative, the base material can be subjected to mechanical energy to free/loosen hydrocarbon fractions from the matrix of the base material. The base material and/or the hydrocarbon rich solvent solution can then be subjected to a separator to separate and withdraw the desired hydrocarbon fractions of certain molecular weights from the base material and/or solution, thereby forming the resultant extraction material. This can involve use of condensation columns, centrifuges, separators, etc. Other mechanical, electrical, and/or chemical systems, in addition to or in lieu of the separator, can be used to facilitate withdrawal of the desired hydrocarbon fractions from the base material and/or the hydrocarbon rich solvent solution.

Hydrocarbon fractions having molecular weights from C1 to C14 can be referred to herein as light hydrocarbon fractions. Hydrocarbon fractions having molecular weights greater than C14 can be referred to herein as heavy hydrocarbon fractions. The extraction process can be used to extract hydrocarbon fractions from the base material having molecular weights from from C1 to C60 (or any range there-between). It is contemplated to utilize the method to more aggressively extract a specific type of hydrocarbons (e.g., light weight hydrocarbons (e.g., C1 to C14) or heavy weight hydrocarbons (e.g., C15 to C60)), because generally material processing applications tend to benefit from having either: 1) material with light hydrocarbons and without heavy hydrocarbons, or 2) material with heavy hydrocarbons and without light hydrocarbons. Other factors may be used that would cause one to utilize the method to more aggressively extract other molecular weight ranges of hydrocarbons. Most conventional systems and methods are not configured to limit the extraction to a specific molecular weight range, but rather attempt to extract all of the hydrocarbon fractions. This is one of the drawbacks of conventional systems, leading to inefficiencies and increased costs.

For instance, with embodiments that are designed to more aggressively extract hydrocarbon fractions from the base material having molecular weights from C1 to C14, the extraction process can be configured to generate a resultant extraction material having hydrocarbon fractions with molecular weights comprising any one or combination of: C1; C1 and/or C2; C1, C2, and/or C3; C1, C2, C3, and/or C4; C1, C2, C3, C4, and/or C5; C1, C2, C3, C4, C5 and/or C6; C1, C2, C3, C4, C5, C6, and/or C7; C1, C2, C3, C4, C5, C6, C7, and/or C8; C1, C2, C3, C4, C5, C6, C7, C8, and/or C9; C1, C2, C3, C4, C5, C6, C7, C8, C9, and/or C10; C1, C2, C3, C4, C5, C6, C7, C8, C9, C10, and/or C11; C1, C2, C3, C4, C5, C6, C7, C8, C9, C10, C11, and/or C12; C1, C2, C3, C4, C5, C6, C7, C8, C9, C10, C11, C12, and/or C13; and/or C1, C2, C3, C4, C5, C6, C7, C8, C9, C10, C11, C12, C13, and/or C14. This can be repeated to extract the hydrocarbon and or bituminous streams until the base material is depleted of hydrocarbons, or can be stopped at any stream from C1-C60 (in a case in which the base material only comprises of hydrocarbons of C1-C60 or a user only wants to deplete the base material of C1-C60 fractions).

The extraction process can involve performing the extraction in iterations. This can involve iteratively extracting hydrocarbon fractions from the base material in stages. For example, a first treatment (e.g., a first heat, chemical, and/or mechanical treatment) can be used to grossly extract light hydrocarbon fractions (e.g., C1-C14), then a second treatment (e.g., a second heat, chemical, and/or mechanical treatment) can be used to more finely extract additional light hydrocarbon fractions, then a third treatment (e.g., a third heat, chemical, and/or mechanical treatment) can be used to even more finely extract additional light hydrocarbon fractions, etc. As another example, a first treatment can be used to extract a first set of light hydrocarbon fractions (e.g., C1-C3), then a second treatment can be used to extract a second set of light hydrocarbon fractions (e.g., C4-C9), then a third treatment can be used to extract a third set of light hydrocarbon fractions (e.g., C10-C14). This iterative process can be done to prevent or reduce the amount of heavy hydrocarbon fractions from being extracted.

For instance, assume the base material has hydrocarbon fractions with molecular weights from C1 to C60, and a user wants to utilize the method to more aggressively extract hydrocarbon fractions from the base material so that the resultant extracted material consists of or consists essentially of hydrocarbon fraction with molecular weights from C1 to C14, thereby leaving the C15 to C60 hydrocarbon fractions behind (leave them in the base material). The extraction process can be configured to generate a resultant extraction material having hydrocarbon fractions with molecular weights comprising any one or combination of: C1; C1 and/or C2; C1, C2, and/or C3; C1, C2, C3, and/or C4; C1, C2, C3, C4, and/or C5; C1, C2, C3, C4, C5 and/or C6; C1, C2, C3, C4, C5, C6, and/or C7; C1, C2, C3, C4, C5, C6, C7, and/or C8; C1, C2, C3, C4, C5, C6, C7, C8, and/or C9; C1, C2, C3, C4, C5, C6, C7, C8, C9, and/or C10; C1, C2, C3, C4, C5, C6, C7, C8, C9, C10, and/or C11; C1, C2, C3, C4, C5, C6, C7, C8, C9, C10, C11, and/or C12; C1, C2, C3, C4, C5, C6, C7, C8, C9, C10, C11, C12, and/or C13; and/or C1, C2, C3, C4, C5, C6, C7, C8, C9, C10, C11, C12, C13, and/or C14. Yet, conventional systems and methods would only be able to extract (or attempt to extract) all of the C1 to C60 hydrocarbon fractions (in a case where the base material only comprises of C1 to C60), and not be able to discriminate the extraction to a desired range of molecular weights.

As another example, assume the base material has hydrocarbon fractions with molecular weights from C1 to C40, and a user wants to utilize the method to more aggressively extract hydrocarbon fractions from the base material so that the resultant extracted material consists of or consists essentially of hydrocarbon fraction with molecular weights from C1 to C10, thereby leaving the C11 to C40 hydrocarbon fractions behind (leave them in the base material). The inventive extraction process can be configured to do this by modulating the treatment of the base material.

As another example, assume the base material has hydrocarbon fractions with molecular weights from C10 to C50, and a user wants to utilize the method to more aggressively extract hydrocarbon fractions from the base material so that the resultant extracted material consists of or consists essentially of hydrocarbon fraction with molecular weights from C to C30, thereby leaving the C31 to C50 hydrocarbon fractions behind (leave them in the base material). The inventive extraction process can be configured to do this by modulating the treatment of the base material.

An exemplary system that can be used to carry out an embodiment of the extraction process can include a heating vessel, a heat source, and a separator. The heating vessel can be a kiln, ladle, crucible, etc. The heat source can be a furnace (e.g., combustion furnace, electric furnace, induction furnace, etc.), heater, heat pump, etc. The separator can be a condenser, columnar condenser, separator, distiller, centrifuge, decanter, etc. Some embodiments can further include fluid displacement mechanism to force or assist the movement of the base material, hydrocarbon rich solvent solution, or resultant extraction material throughout the system. This can include a pump, a paddle, a propeller, etc.

For instance, the system can include a heating vessel configured to contain base material and/or solvent that will be heated. The heating vessel can be connected to, positioned proximate to, or placed within the heating source. The heating vessel can be connected to the separator so that vapors and volatiles driven off by the heating process are directed from the heating vessel to the separator. The vapors and volatiles contain the hydrocarbon fractions within the desired range of molecular weights to be extracted (e.g., the C1 to C2, C1 to C3, C1 to C4, and so on). Adjustment of the heating treatment and/or the solvent used can be done to adjust the molecular weights of hydrocarbon fractions that will be in the vapors and volatiles, or present in the extraction medium such as water or volatile solvent. The separator can be configured to separate out the desired hydrocarbon fractions from other components. At least one fluid displacement mechanism can be connected to a portion of the system to force or assist the movement of base material, hydrocarbon rich solvent solution, and/or resultant extraction material.

In a non-limiting, exemplary operation of the system, base material can be placed inside the heating vessel. The heating vessel can be placed on, at, near, or within the heating source so that heat is transferred to the base material. The heating vessel and/or separator can be configured to prevent any vapors and volatiles being driven off from the base material to flow from the heating vessel until permitted to do so. This can be achieved via the use of valves, for example. Thus, the system can operate under heating campaigns. A heating campaign can be subjecting the base material (and solvent if a solvent is used) to a heating treatment. The heating treatment can include subjecting the base material and/or solvent to a predetermined amount of heat (a predetermined temperature or a predetermined range of temperatures) for a predetermined time duration.

Increasing any one or combination of the temperature and the time duration can increase the amount of hydrocarbon fractions that become free. In addition, increasing any one or combination of the temperature and the time duration can increase the proportional amount of light hydrocarbon fractions that become free. Naturally, increasing these operating parameters can increase the costs associated with operating the system, and thus a cost-benefit analysis can be performed. Thus, the heating campaign can be adjusted to adjust the amount and/or molecular weight of hydrocarbon fraction material to be extracted. For instance, the greater the temperature, and the time duration used for the heating campaign, the greater the amount and the greater the molecular weight of hydrocarbon fraction material is driven off as vapor or volatiles. As can be appreciated, one can perform a cost-benefit analysis to determine the optimal heating campaign that would result in a maximum amount of desired molecular weight hydrocarbon fraction material at the minimal cost.

The vapor or volatiles generated during the heating treatment can be directed to the separator. As noted herein, some embodiments use a solvent to generate a solvent solution for, and thus the vapor or volatiles can include a hydrocarbon rich solvent solution. An embodiment of the separator can be configured as a condenser having a tube (inner tube) within a tube (outer tube). The vapor or volatiles can be directed through the inner tube, while coolant (e.g., H₂O) is circulated throughout the outer tube. The coolant can cause the vapor or volatiles to cool and condense, which can condense to a liquid. This liquid can contain the resultant extracted material. The types of hydrocarbon fractions (e.g., light, heavy, etc.) and the relative amounts of hydrocarbon fractions within the resultant extracted material will be a function of the base material used, the solvent used, and the operating parameters of the heating treatment.

It should be noted that embodiments of the system and method can be operated without any application of pressure (positive or negative) in the system. While embodiments of the system may be configured to utility pressure, no pressure or vacuum is necessary for effective use of the system. For instance, the vapor and volatiles are driven up through the separator and cool and condense before reaching any vent or opening in the separator. The condensed vapors and volatiles are then collected. Thus, no pressure if necessary for proper and effective operation of the system. This significantly reduces costs and increases safety, and is in stark contrast to many conventional systems. In addition, because no vapor or volatiles reach the vent, none of the hydrocarbon fractions have to be vented off (or otherwise escape the system) or flared off. This significantly reduces environmental liability, and is in stark contrast to most conventional systems.

As a non-limiting example, the system can be operated at 350° F. for 30 minutes to generate a resultant extracted material having a 25% hydrocarbon extraction yield by weight of hydrocarbon fractions (i.e., if 100 grams of base material is put in the heating vessel, 25 grams of hydrocarbon fractions can be extracted). Thus, the hydrocarbon extraction yield at these operating parameters can be 25%. Test results on this resultant extracted material reveal that 70% of these 25 grams of hydrocarbon fractions are within the range of C1 to C20, and 30% of these 25 grams of hydrocarbon fractions are greater than C20. This type of yield can be referred to as light hydrocarbon fraction extraction yield. Even though light hydrocarbon fractions are defined herein as being within the range from C1 to C14, increasing the percentage of C1 to C20 hydrocarbons in the extracted material will increase the amount of C1 to C14 hydrocarbons, thereby increase the light hydrocarbon extraction yield. As noted above, the heating campaign can be adjusted to adjust the amount and/or molecular weight of the hydrocarbon fractions within the resultant extracted material. Thus, operating temperatures greater than 350° F. and at time durations greater than 30 minutes can result in greater than 25% hydrocarbon extraction yield and/or greater than 70% light hydrocarbon fraction extraction yield.

Another exemplary system that can be used to carry out an embodiment of the extraction process can include a particle collider. The particle collider can receive the base material, wherein one or more high pressure pumps can force streams of the base material into each other. This collision is an example of imparting mechanical energy into the base material.

Another technique that can be used to adjust the hydrocarbon extraction yield and/or the light hydrocarbon fraction extraction yield can be adjusting the mix used as the base material. Some base materials can be dryer than others. A mixture comprising a combination of a less dry base material and a more dry base material can be used to further adjust the hydrocarbon extraction yield and/or the light hydrocarbon fraction extraction yield. For instance, a greater hydrocarbon extraction yield and/or light hydrocarbon fraction extraction yield can be obtained from a base material that comprises a mixture wet and dry base material, as opposed to a base material consisting of wet material only or consisting of dry material only. Without wishing to being limited by theory, it is hypothesized that the mixture provides improved yields because the lighter hydrocarbon fractions in the less dry base material serve to loosen the hydrocarbon fractions in the more dry base material, thereby acting as a solvent for the mixture.

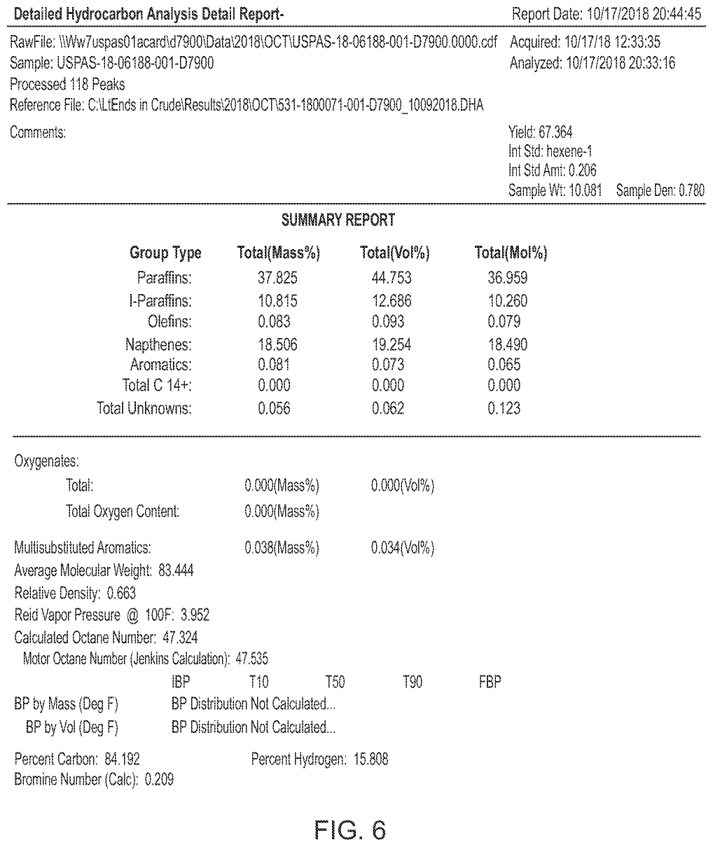

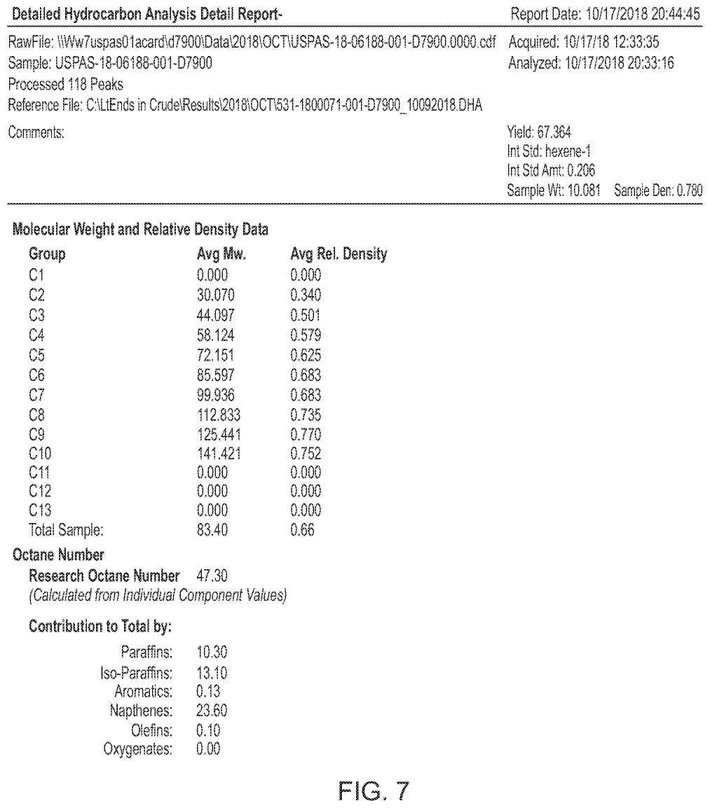

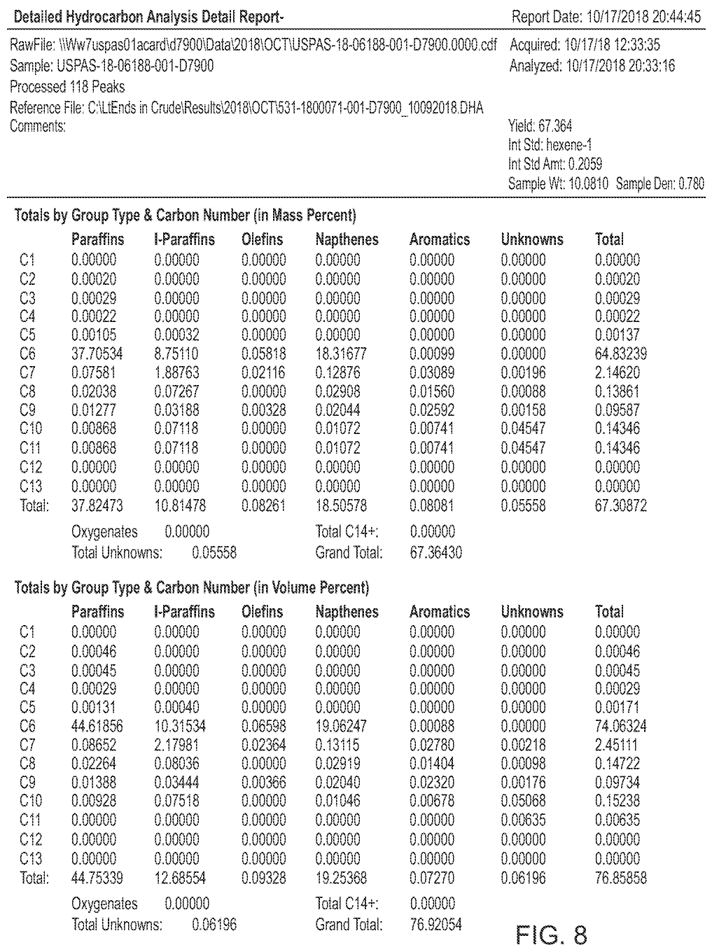

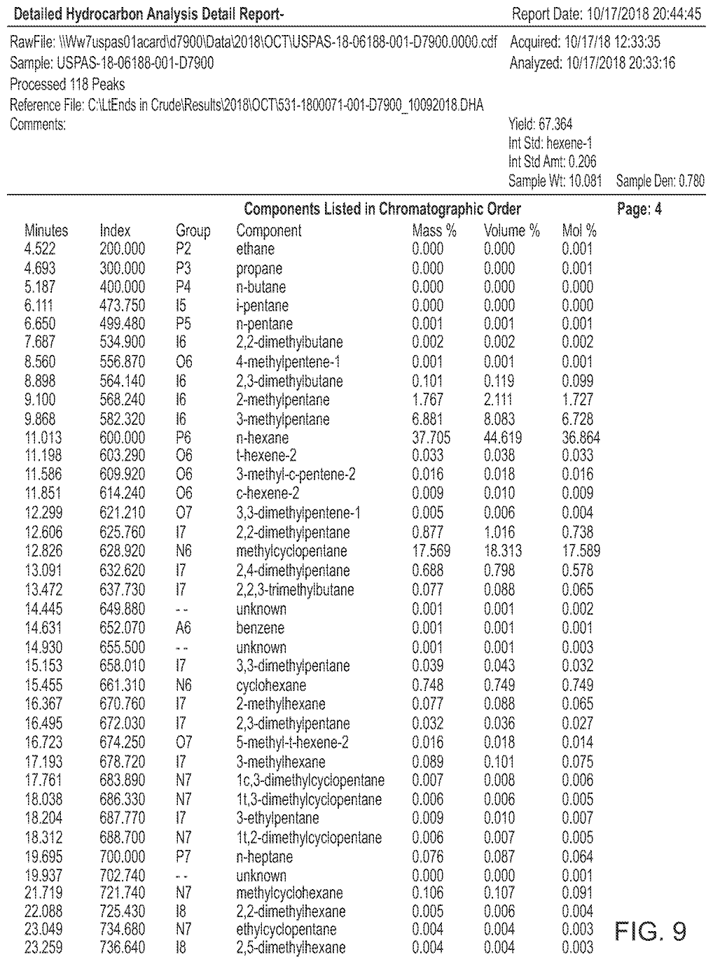

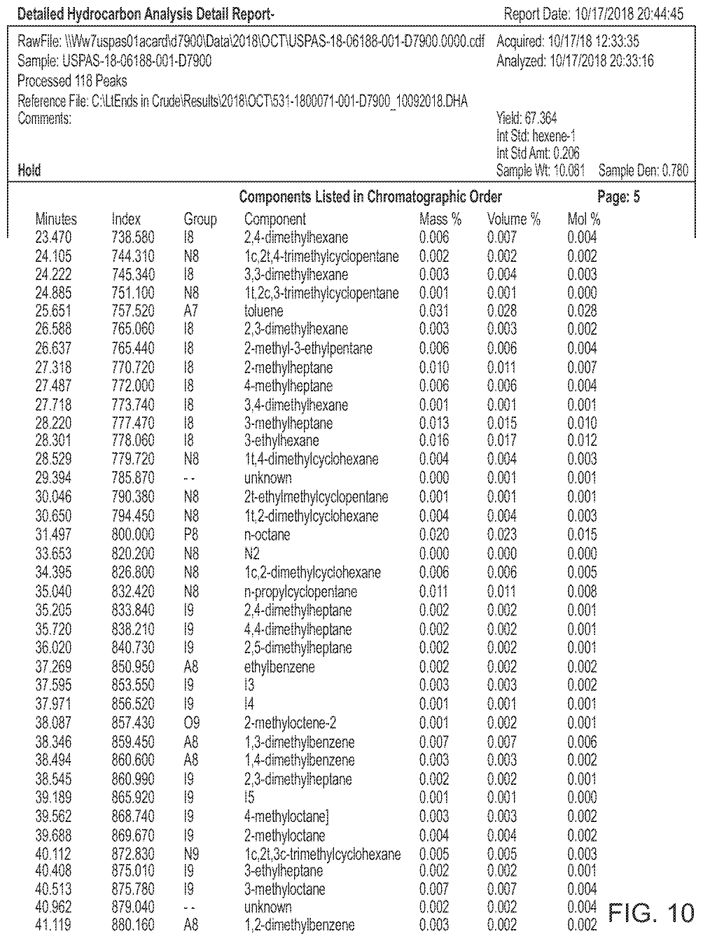

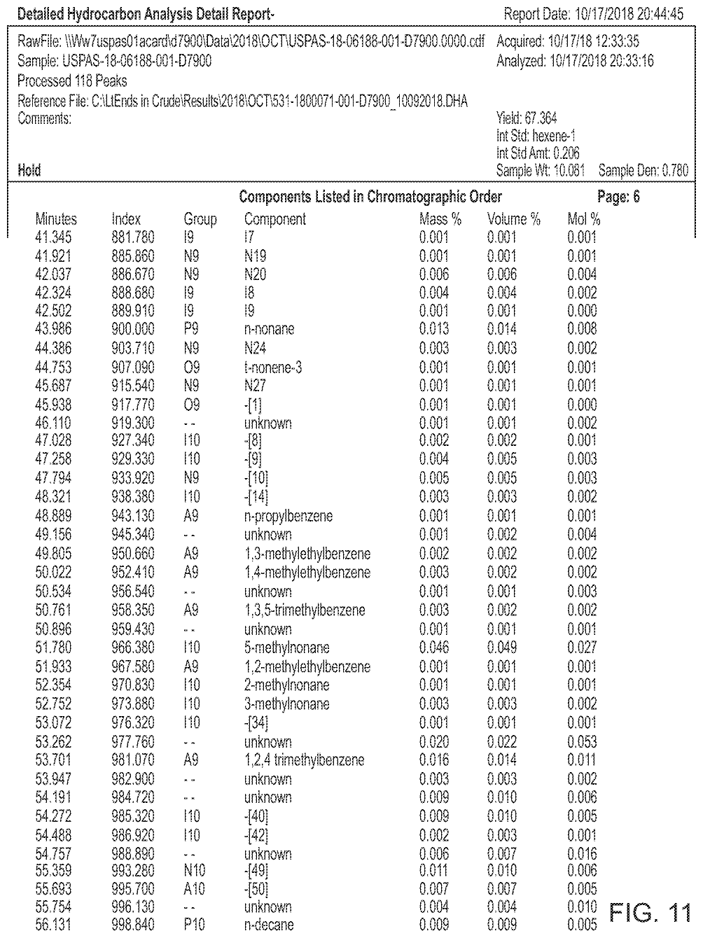

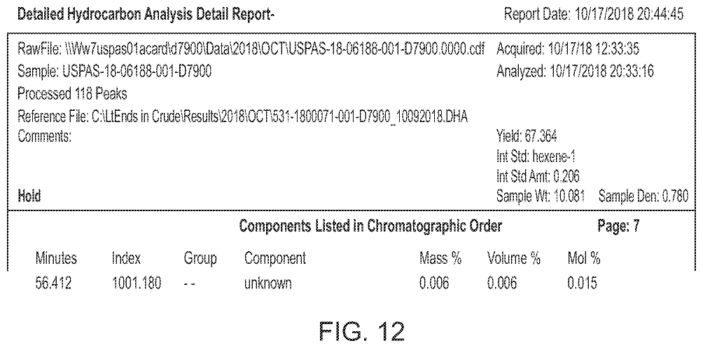

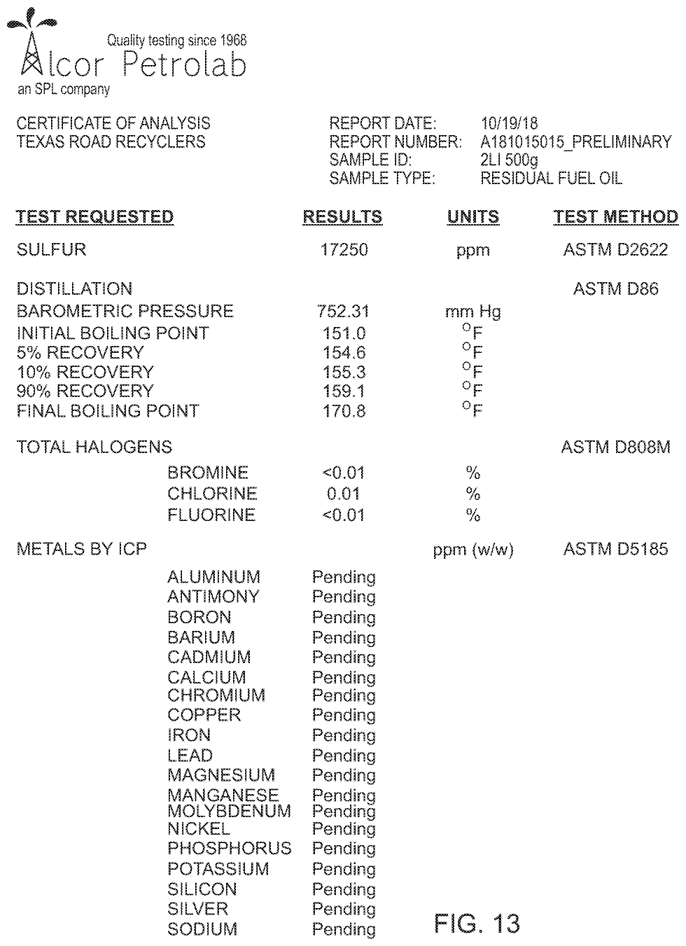

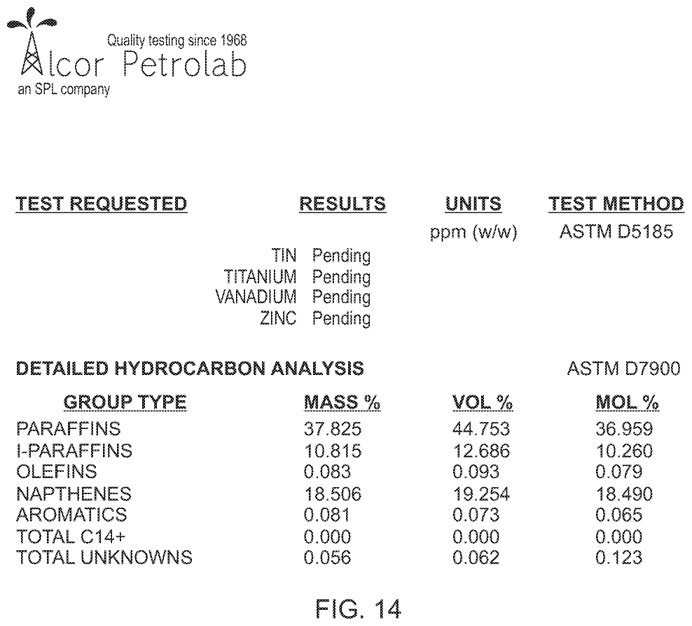

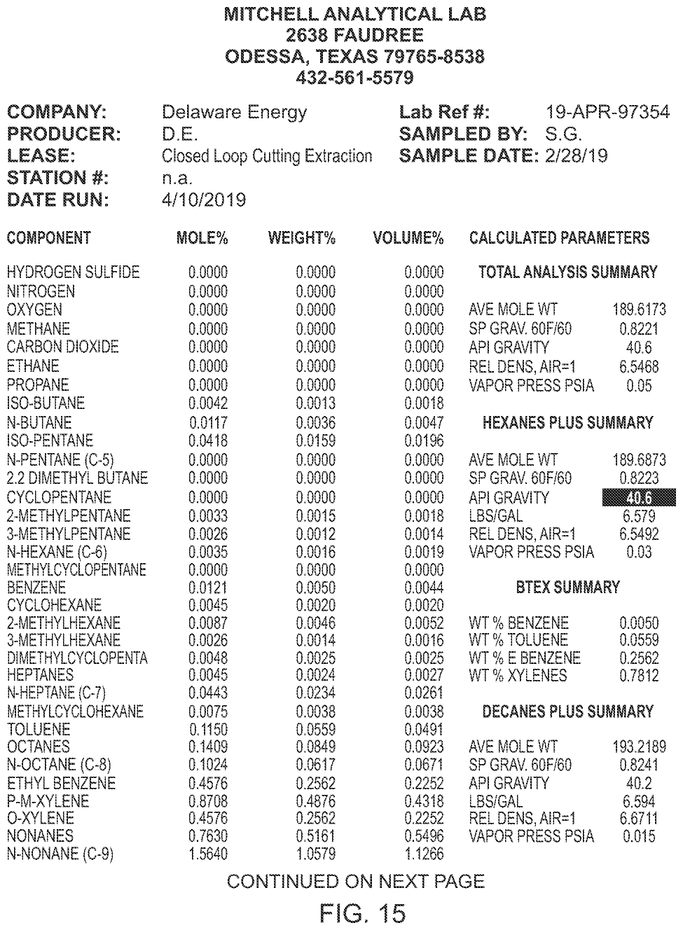

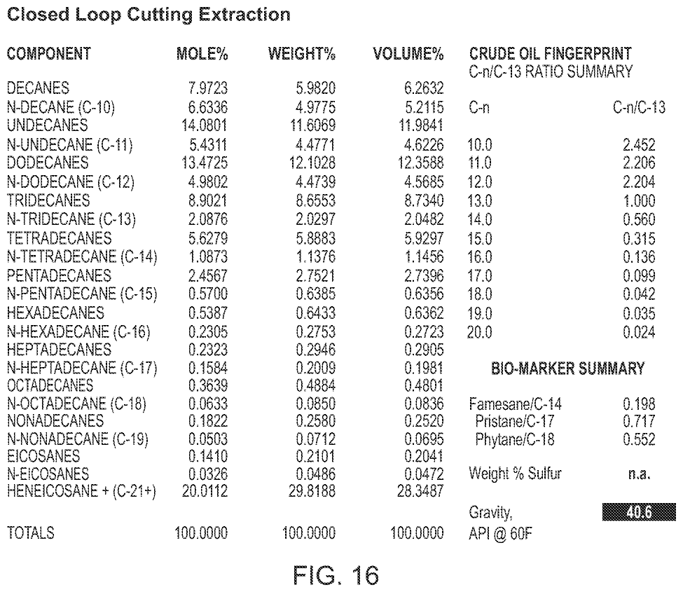

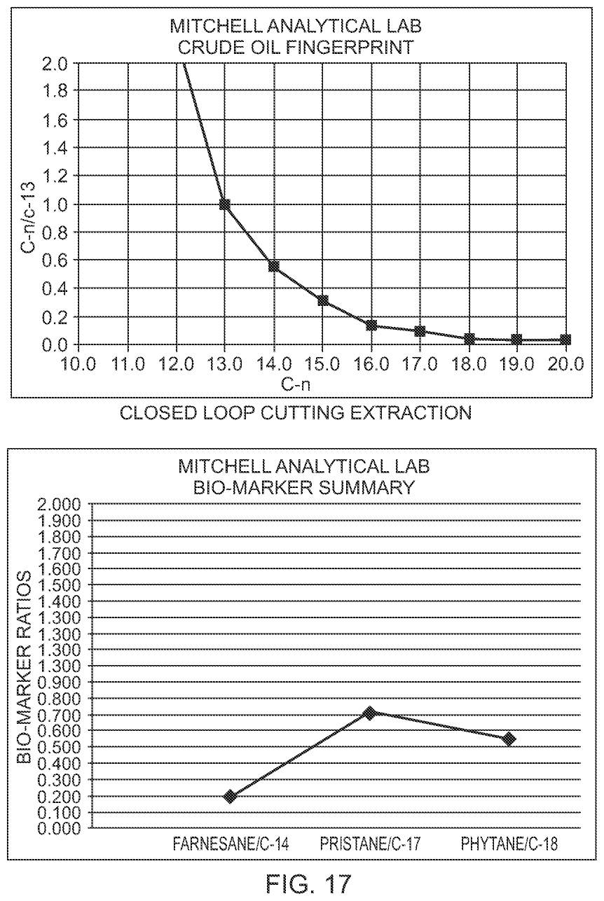

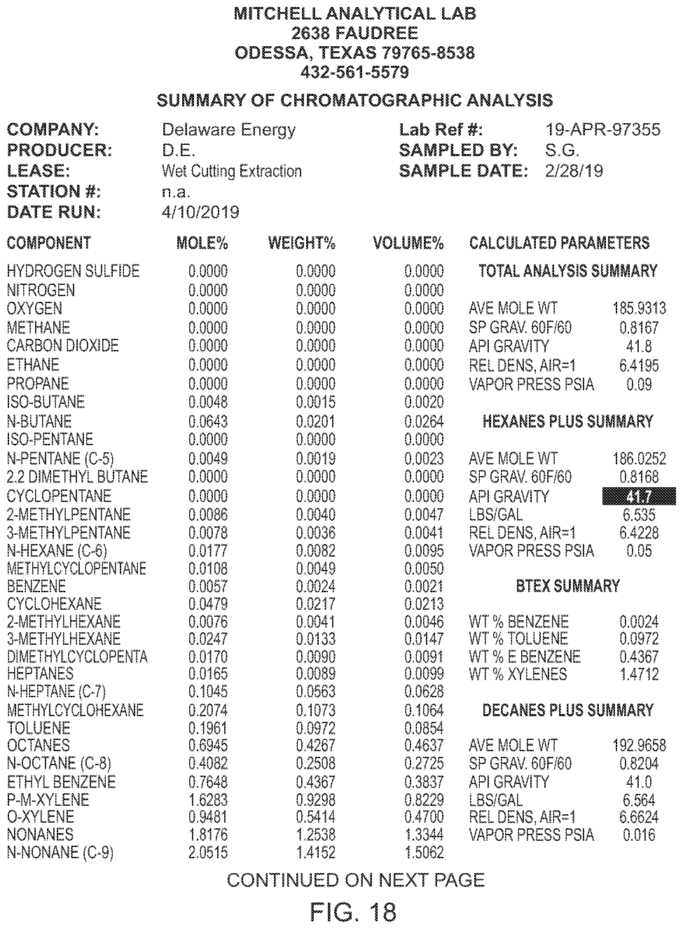

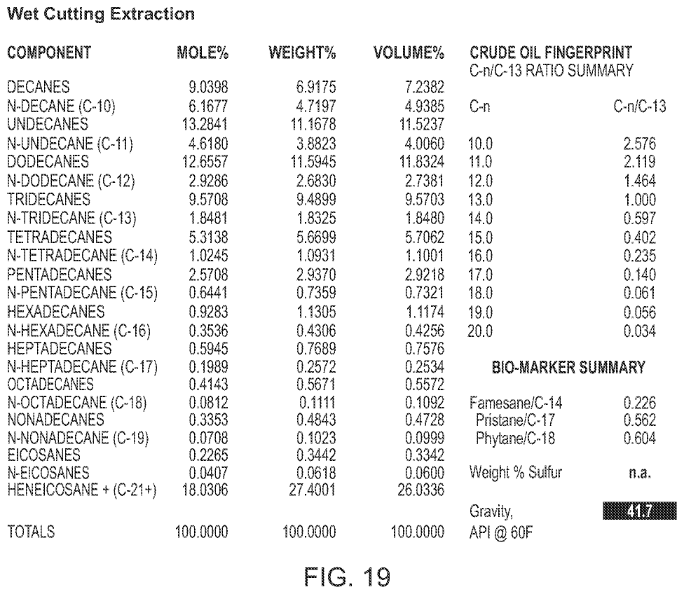

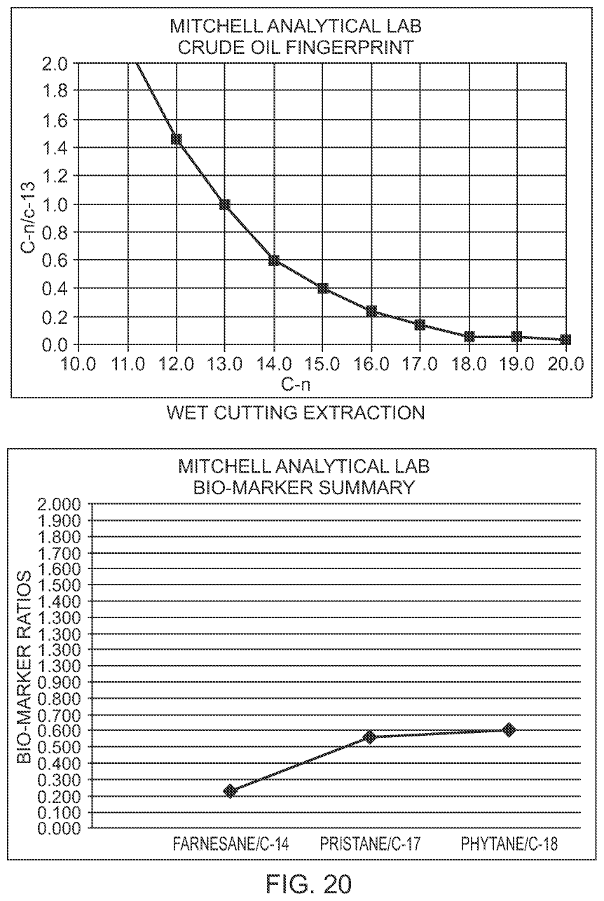

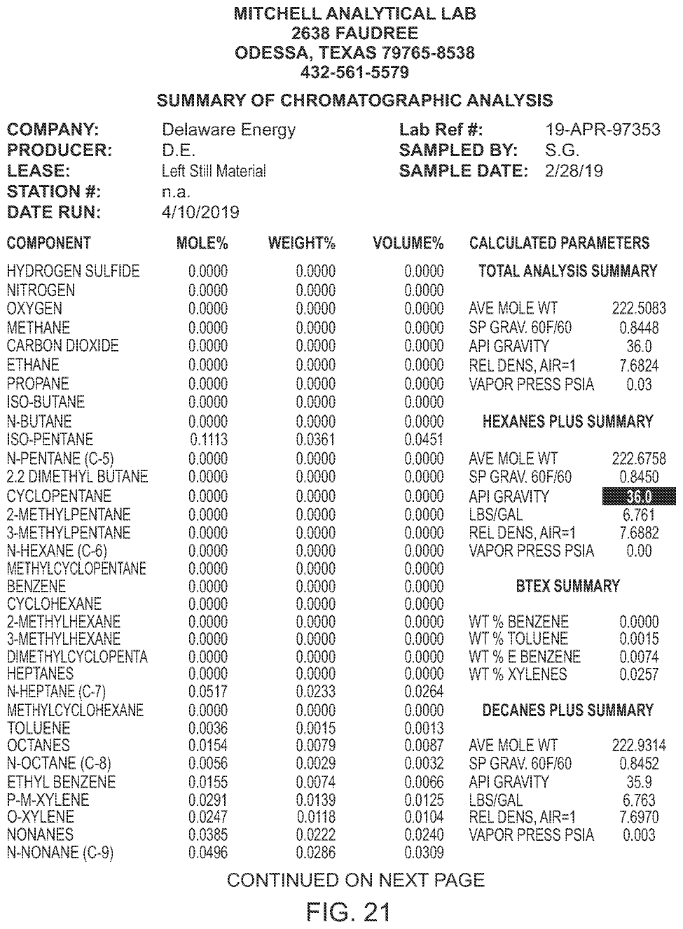

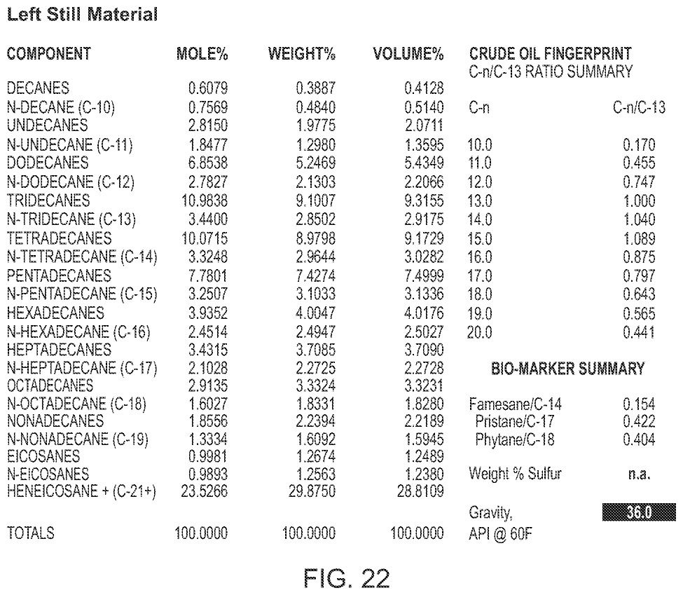

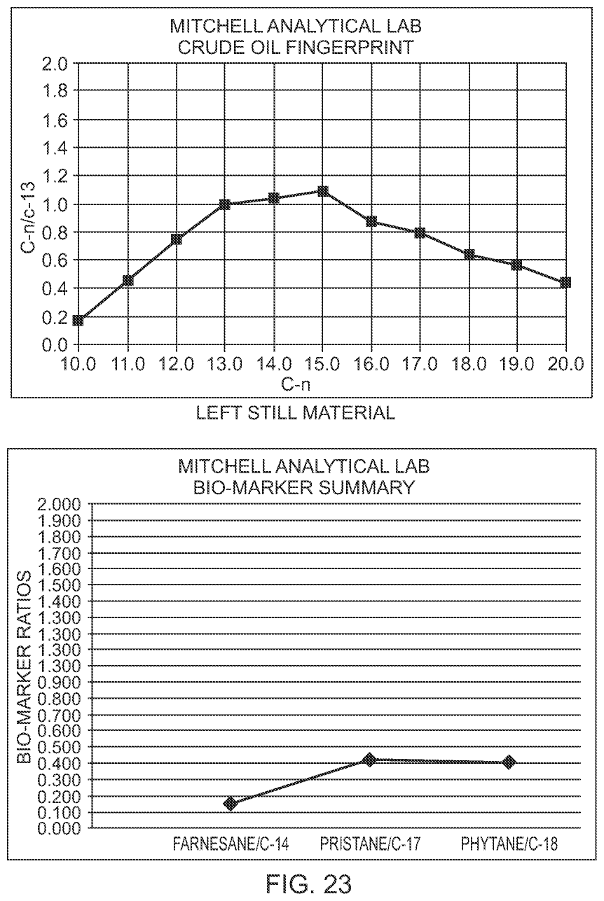

FIGS. 6-14 are laboratory test results of resultant extracted material that has been generated using an embodiment of the extraction process. FIGS. 15-17 are laboratory test results of resultant extracted material, showing chromatographic analyses, crude oil fingerprint, and bio-marker summary information for drill cuttings obtained from closed loop extraction drilling methods (closed loop extraction generates dryer material). FIGS. 18-20 are laboratory test results of resultant extracted material, showing chromatographic analyses, crude oil fingerprint, and bio-marker summary information for drill cuttings obtained from wet cutting extraction drilling methods (wet cutting extraction generates wetter material). FIGS. 21-23 are laboratory test results of resultant extracted material, showing chromatographic analyses, crude oil fingerprint, and bio-marker summary information for left still material.

As noted herein, the extracted material can be useful in other processes. However, the extracted material can also be further processed to retrieve useful components therefrom—i.e., not only can the extracted material be useful in other processes, but itself also contains components that can be further extracted therefrom. For instance, the extracted material can have hydrocarbon fractions (these can be light, heavy, or both depending on the methods used), sulfur compounds, other compounds, minerals, elements, free carbons (e.g., graphene) atoms, etc. that may be useful for other purposes. Some bituminous material also contains these materials. Thus, the retrieval processes disclosed below can be applied to the bituminous material and/or the extracted material to retrieve these components.

As noted herein, which type of treatment to used (e.g., mechanical, chemical, or heat), to which degree and/or duration to use it/them (e.g., some treatments tend to destroy certain hydrocarbon fractions), if and when to use a combination of them, whether to use it/them in iterations, etc. can be selected to optimize the material properties of the conditioned base material or extracted material, optimize process operations that are used to produce the same, and/or optimize process operations to process the conditioned base material and/or the extracted material after being produced.

It should be understood that modifications to the embodiments disclosed herein can be made to meet a particular set of design criteria. For instance, the number of or configuration of process steps and/or operating parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

Therefore, it is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of apparatuses and methods of making and using the same have been discussed and illustrated herein, it is to be distinctly understood that the invention is not limited thereto but may be otherwise variously embodied and practiced within the scope of the following claims.

What is claimed is:

1. A method for extracting hydrocarbon fractions from a material, the method comprising:
subjecting a base material comprising hydrocarbon fractions to an extraction process, the extraction process involving a treatment configured to free or loosen hydrocarbon factions from a matrix of the base material, the treatment generating volatiles comprising hydrocarbon fractions within a desired range of molecular weights; and
allowing the volatiles to enter a separator for separating the hydrocarbon fractions having molecular weights with the desired range of molecular weights from other components of the volatiles to generate a resultant extracted material.

2. The method of claim 1, wherein the treatment involves a heat treatment, a mechanical treatment, and/or a chemical treatment to impart energy to the base material for freeing or loosening the hydrocarbon fractions.

3. The method of claim 1, wherein the base material includes Limestone Rock Aggregate (“LRA”).

4. The method of claim 1, wherein the base material consists essentially of Limestone Rock Aggregate (“LRA”).

5. The method of claim 1, wherein the base material consists of Limestone Rock Aggregate (“LRA”).

6. The method of claim 1, wherein the base material includes Limestone Rock Aggregate (“LRA”) crusher fines.

7. The method of claim 1, wherein the base material consists essentially of Limestone Rock Aggregate (“LRA”) crusher fines.

8. The method of claim 1, wherein the base material consists of Limestone Rock Aggregate (“LRA”) crusher fines.

9. The method recited in claim 1, further comprising adjusting the treatment to modulate the desired range of molecular weights.

10. The method recited in claim 1, further comprising performing the extraction process in iterations to modulate the desired range of molecular weights.

11. The method recited in claim 1, wherein:
the base material comprises hydrocarbon fractions having molecular weights within a first range;
the resultant extracted material comprises hydrocarbon fractions having molecular weights within a second range; and
the first range is greater than the second range.

12. The method recited in claim 11, wherein the first range is from C1 to C60.

13. The method recited in claim 11, wherein the second range is from C1 to C14.

14. The method recited in claim 2, further comprising modulating the desired range of molecular weights by adjusting an amount and a duration of energy imparted to the base material.

15. The method recited in claim 2, further comprising modulating the desired range of molecular weights by selecting two or more of the heat treatment, the mechanical treatment, and the chemical treatment to be part of the treatment.

16. The method recited in claim 15, further comprising modulating the desired range of molecular weights by using one form of treatment before the other form of treatment.

17. The method recited in claim 2, further comprising modulating the desired range of molecular weights by:
selecting two or more of the heat treatment, the mechanical treatment, and the chemical treatment to be part of the treatment;
using one form of treatment before the other form of treatment; and
adjusting an amount and a duration of energy imparted to the base material.

18. The method recited in claim 17, further comprising plural modulation techniques.

19. The method recited in claim 18, wherein:
a first modulation technique targets a first molecular weight or a first range of molecular weights; and
a second modulation technique targets a second molecular weight or a second range of molecular weights.

20. The method recited in claim 19, wherein:
the first modulation technique frees or loosens a first amount of hydrocarbon fractions; and
the second modulation technique frees or loosens a second amount of hydrocarbon fractions;
wherein the first amount of hydrocarbon fractions is different from the second amount of hydrocarbon fractions.
